(19) United States
(12) Patent Application Publication
(10) Pub. No.: US 2024/0277220 A1
(43) Pub. Date: Aug. 22, 2024

(54) MEDICAL VISUALISATION DEVICE WITH PROGRAMMABLE BUTTONS

(71) Applicant: AMBU A/S, Ballerup (DK)

(72) Inventors: Morten Grønning NIELSEN, Copenhagen N (DK); Line Sandahl UBBESEN, Holte (DK)

(73) Assignee: AMBU A/S, Ballerup (DK)

(21) Appl. No.: 18/566,703

(22) PCT Filed: Jun. 13, 2022
(86) PCT No.: PCT/EP2022/066028

§ 371 Date: Dec. 4, 2023

(30) Foreign Application Priority Data
Jun. 15, 2021 (DK) PA2021 70302

(51) Int. Cl. A61B 1/267; A61B 1/00; A61B 1/05

(52) U.S. Cl. CPC A61B 1/267; A61B 1/00006; A61B 1/0004; A61B 1/00045; A61B 1/00071; A61B 1/05

(57) ABSTRACT

A medical visualization system including a monitor device are disclosed. The monitor device includes a housing and a processing unit adapted to receive image data from a medical visualization device and cause a display to display a live representation of the image data with a first portion of a graphical user interface, A representation of a medical visualization device includes a first programmable button. The monitor device is adapted to detect a first user input corresponding to selection of the first programmable button, and in response to detecting the first user input the monitor device displays, within a second portion of the graphical user interface, a first button setup menu for selecting actions to be associated with activations of the first programmable button.

The present disclosure relates to a medical visualization system and elements thereof. Particularly, the present disclosure relates to a monitor device for medical visualization devices, such as endoscopes, laryngoscopes etc., in particular to a monitor device comprising a touch sensitive display and for medical visualization devices having programmable buttons. More particularly, the present disclosure relates to programming of such buttons of a medical visualization device.

### BACKGROUND

A medical visualization device may be utilized to visually examine certain areas of the body of a person, such as inside a body cavity of the person. For example, a medical visualization device may be used to inspect the airways, the digestive tract, or the intestines.

A medical visualization device may be provided with a camera and be attached to a monitor device, such as a monitor with a display screen, a video output from the camera of the visualization device may be received and displayed at the monitor device, thereby allowing an operator to control the visualization device to inspect an area of interest.

For example, a visualization device may be an endoscope, such as a disposable endoscope. An endoscope comprises an operating handle at the proximal end and an insertion cord extending from the handle towards a distal end. The handle is configured to be held by an operator and inter alia comprises externally protruding operating members connected to internal control means allowing the operator to control the movement of a bending section near the distal end of the insertion cord, while advancing the distal end of the insertion cord to a desired location e.g. within a body cavity of a person. By means of an attached monitor device, such as a monitor with a display screen, the location to which the distal end has been advanced may be inspected using the endoscope.

The monitor device of a medical visualization system may be provided with some functionality, such as ability to save still images and/or video sequences of the view from the attached visualization device. Furthermore, the monitor device may comprise some image processing capabilities, and may be configured to output a video or image output, e.g. to an external display.

Some endoscope systems may comprise buttons for taking images or video etc. However, programming of such buttons may be either unavailable or may be a tedious task.

### SUMMARY

It is an object of the present disclosure to provide a solution which at least improve the solutions of the prior art. Particularly, it is an object of the present disclosure to provide a graphical user interface for a medical visualization system which facilitates and enhances human interaction with the system. Particularly, it is an object of the present disclosure to provide solutions which facilitates easy programming of buttons provided on a medical visualization device.

The present disclosure relates to a monitor device operable to receive image data from one or more medical visualization devices, and a medical visualization system comprising the monitor device. The present disclosure further relates to a graphical user interface for such monitor device.

Accordingly, a monitor device is disclosed, wherein the monitor device is operable to receive image data from one or more medical visualization devices. The medical visualization devices may be coupled or couplable to the monitor device.

The monitor device comprises a housing, and a processing unit. The processing unit is adapted to receive the image data from the medical visualization device and cause a display, e.g. a touch sensitive display, to display a live representation of the image data. The display may be a display of the monitor device, or an external display coupled to the monitor device.

The monitor device displays with the display a graphical user interface. The monitor device displays, within a first portion of the graphical user interface, a representation of a medical visualization device comprising one or more programmable buttons including a first programmable button.

The monitor device is adapted to detect a first user input corresponding to selection of the first programmable button. In response to detecting the first user input the monitor device displays, within a second portion of the graphical user interface, a first button setup menu for selecting one or more actions to be associated with one or more activations of the first programmable button.

Also, a medical visualization system is disclosed. The medical visualization system comprises the disclosed monitor device. The medical visualization system may comprise a medical visualization device. The medical visualization device comprises: an image sensor adapted to generate image data indicative of a view from the medical visualization device, and a handle having one or more programmable buttons. The medical visualization device may further comprise a light emitter adapted to provide illumination of the view.

By the present disclosure, the user may easily adjust the functionality assigned to each of one or more programmable buttons of a medical visualization device. The disclosure further makes it clear and easy of the user to realize which type of visualization device is being configured even in a system, which may be used for different types of medical visualization device, which again may be used for very different clinical purposes.

### BRIEF DESCRIPTION OF THE FIGURES

Embodiments of the disclosure will be described in more detail in the following with regard to the accompanying figures. The figures show one way of implementing the present disclosure and are not to be construed as being limiting to other possible embodiments falling within the scope of the attached claim set.

FIG. 1 schematically illustrates an exemplary medical visualization system,

FIG. 2 schematically illustrates an exemplary monitor device,

FIG. 3 schematically illustrates an exemplary monitor device,

FIG. 4 is a block diagram of an exemplary monitor device,

FIGS. 5A-5F schematically illustrates exemplary user interactions with an exemplary graphical user interface,

FIGS. 6A-6D schematically illustrates exemplary user interactions with an exemplary graphical user interface,

FIGS. 7A-7C schematically illustrates exemplary user interactions with an exemplary graphical user interface, and

FIGS. 8A and 8B schematically illustrates exemplary part of a graphical user interface.

### DETAILED DESCRIPTION

The medical visualization device may be an endoscope. Particularly, but not exclusively the medical visualization device may be a disposable camera endoscope. Alternatively, the medical visualization device may be a laryngoscope, an endotracheal tube. The medical visualization device may be a disposable, e.g. single use, medical visualization device.

The medical visualization device may comprise an insertion cord extending from the handle to a distal cord portion. The view from the visualization device may be a view from the distal cord portion of the insertion cord.

The handle may comprise a control mechanism adapted to receive an input in a first input direction and/or in a second input direction. The input in the first input direction may cause a bendable section of the insertion cord to bend in a first bending direction. The input in the second input direction may cause the bendable section of the insertion cord to bend in a second bending direction. The second bending direction may be opposite the first bending direction.

The housing of the monitor device may be a first housing and/or a second housing. The first housing may accommodate the display, which may be a touch sensitive display. The second housing may be void of a display.

The first housing may extend in a first direction from a first housing side to a second housing side and in a second direction perpendicular to the first direction from a third housing side to a fourth housing side. The display, such as the touch sensitive display, may be accommodated in the first housing. The display may have a first length in the first direction and a second length in the second direction. The second length may be longer than the first length, e.g. the display may be a 16:9 or 16:10 display. Alternatively, the first length may be longer than the second length, or the first length and the second length may be substantially the same. The touch sensitive display may be any suitable type of touch display, e.g. capacitive touch display or resistive touch display.

The monitor device may comprise one or more connection ports configured to receive a connector of the visualisation device. The connection ports and the corresponding connector of the visualisation device may be a proprietary plug and socket connectors, or any standard connector capable of transmitting therethrough at least the image data from the image sensor. Furthermore, the connector and connection ports may be configured to supply power to the components of the visualisation device.

The one or more connection ports may be arranged on the first housing. The one or more connection ports may be provided on the third housing side, and/or on the fourth housing side. Alternatively or additionally, the one or more connection ports may be arranged on the second housing.

The monitor device may comprise an on/off button. The on/off button may be arranged on the first housing and/or on the second housing. The on/off button may be provided on the third housing side or on the fourth housing side of the first housing. The one or more connection ports may be provided on the third housing side and the on/off button may be provided on the fourth housing side of the first housing.

The monitor device may establish connection to a medical visualisation device. Establishing connection to the medical visualisation device may include receiving a device connector of the respective visualisation device in a connection port of the one or more connection ports of the monitor device. Establishing connection to a medical visualisation device may include obtaining device identifier information from a device identifier (e.g. EPROM, QR-code, NFC, RFID or similar) of the medical visualisation device. For example, establishing connection to a first medical visualisation device may include obtaining first device identifier information from a first device identifier of the first medical visualisation device and/or establishing connection to a second medical visualisation device may include obtaining second device identifier information from a second device identifier of the second medical visualisation device.

The monitor device may comprise a processing unit and memory. The processing unit and/or the memory may be accommodated in the first housing or in the second housing. In some examples, the processing unit and/or the memory may be accommodated in the second housing and the display may be accommodated in the first housing.

The monitor device may comprise an orientation sensor, e.g. for determining the orientation of the monitor device, such as of the first housing, relative to gravity. The orientation sensor may comprise one or more accelerometers and/or a gyroscope. The orientation sensor may be accommodated in the first housing. The processing unit may be connected to the orientation sensor to receive an orientation signal indicative of the orientation of the monitor device, such as of the first housing of the monitor device.

The processing unit may be connected to the display to control display of information with the display. The processing unit may be adapted to receive a signal from the display, particularly when the display is a touch sensitive display, indicative of touch inputs on the display. Thereby, the monitor device may detect user inputs, e.g. in the form of touch inputs, with the touch sensitive display. Alternatively, the monitor device may detect user inputs by means of a keyboard, a trackpad, a mouse etc. Touch inputs may, for example, comprise single tap(s), double tap(s), or swipe(s) on the touch sensitive display.

The processing unit may be connected to the memory and be adapted to read and write data from and to the memory.

The monitor device may comprise a power unit for powering the monitor device. The power unit may comprise a rechargeable battery and/or a power connection for connecting the power unit to an external power supply, such as a conventional AC power socket. The power unit may be accommodated in the first housing. Alternatively, the power unit may be accommodated in the second housing.

The monitor device may comprise the graphical user interface. The monitor device and/or the processing unit of the monitor device may display with the display the graphical user interface. The graphical user interface may comprise one or more portions, such as a plurality of portions, e.g. including the first portion and the second portion, and optionally a third portion and/or a fourth portion. The portions may be non-overlapping portions, such as a plurality of non-overlapping portions. Each of the plurality of portions may extend substantially throughout the first length in the first direction. The first portion may be arranged between the third portion and the second portion along the second direction. The third portion may be arranged between the fourth portion and the first portion along the second direction. The fourth portion may be arranged between a side of the first housing, e.g. the third housing side, and the third portion along the second direction. The second portion may be arranged between another side of the first housing, e.g. the fourth housing side, and the first portion along the second direction. The first portion and the third portion may be arranged between the second portion and the fourth portion along the second direction. The first portion of the graphical user interface may be square. The first portion of the graphical user interface may occupy the centre of the display. The first portion of the graphical user interface may be larger along the second direction than the second portion, the third portion and/or the fourth portion, individually and/or collectively. The first portion of the graphical user interface may extend throughout more than 40% of the second length in the second direction, such as more than 50% of the second length in the second direction, such as more than 60% of the second length in the second direction.

The medical visualization device may, as stated previously, comprise a control mechanism adapted to receive an input in a first input direction and/or in a second input direction, and wherein the input in the first input direction may cause a bendable section of the insertion cord to bend in a first bending direction, and wherein the input in the second input direction may cause the bendable section to bend in a second bending direction. The live representation of the image data may have directions corresponding to directions of the image sensor generating the image data. The first bending direction may correspond to a first image direction of a representation of the image data, such as the live representation of the image data. The second bending direction may correspond to a second image direction of the representation of the image data, such as the live representation of the image data. The first image direction and/or the second image direction may be parallel to the first direction of the first housing.

The monitor device displays, within the first portion of the graphical user interface, a representation of a medical visualization device comprising one or more programmable buttons including a first programmable button.

The monitor device is adapted to detect a first user input corresponding to selection of the first programmable button. In response to detecting the first user input the monitor device displays, within the second portion of the graphical user interface, a first button setup menu for selecting one or more actions to be associated with one or more activations of the first programmable button.

The one or more programmable buttons of the medical visualization device may further include a second programmable button. The monitor device may be adapted to detect a second user input corresponding to selection of the second programmable button. In response to detecting the second user input the monitor device may display, within the second portion of the graphical user interface, a second button setup menu for selecting one or more actions to be associated with one or more activations of the second programmable button.

The first user input and/or the second user input may be detected with the display, e.g. when the display is a touch sensitive display. Alternatively or additionally, the first user input and/or the second user input may be detected with a mouse, a trackpad and/or a keyboard, which may be coupled with the monitor device.

The first button setup menu may comprise a first list of actions. The second button setup menu may comprise a second list of actions. The first list of actions and the second list of actions comprises the same actions. Hence, the user may be able to assign the same action to different buttons. Also, the lists comprising the same actions facilitates easy selection for the user.

The one or more activations of a programmable button, such as the one or more activations of the first programmable button and/or of the second programmable button, may include a plurality of activations, e.g. a short press of the programmable button and a long press of the programmable button.

The first button setup menu may comprise a first short press menu option for selecting a first primary action to be associated with the short press of the first programmable button and a long press menu option for selecting a first secondary action to be associated with the long press of the first programmable button. The second button setup menu may comprise a second short press menu option for selecting a second primary action to be associated with the short press of the second programmable button and a long press menu option for selecting a second secondary action to be associated with the long press of the second programmable button.

The short press of a programmable button, such as the first programmable button or the second programmable button may correspond to an activation of the programmable button for a duration being less than a threshold duration. The long press of the programmable button may correspond to an activation of the programmable button for a duration being longer than the threshold duration. The threshold duration may be between 0.5-1.5 seconds, such as 1 second.

The first short press menu option may comprise a first primary list of actions. Selecting the first primary action to be associated with the short press of the first programmable button may include selecting the first primary action from the first primary list of actions.

The first long press menu option may comprise a first secondary list of actions. Selecting the first secondary action to be associated with the long press of the first programmable button may include selecting the first secondary action from the first secondary list of actions. The first primary list of actions and the first secondary list of actions may comprise the same actions. Hence, the user may be able to assign the same action both to a long press and a short press of the respective button. Also, the lists comprising the same actions facilitates easy selection for the user.

The first list of actions, as mentioned previously, may comprise one or both of the first primary list of actions and/or the first secondary list of actions.

The second short press menu option may comprise a second primary list of actions. Selecting the second primary action to be associated with the short press of the second programmable button may include selecting the second primary action from the second primary list of actions.

The second long press menu option may comprise a second secondary list of actions. Selecting the second secondary action to be associated with the long press of the second programmable button may include selecting the second secondary action from the second secondary list of actions. The second primary list of actions and the second secondary list of actions may comprise the same actions. Hence, the user may be able to assign the same action both to a long press and a short press of the respective button. Also, the lists comprising the same actions facilitates easy selection for the user.

The second list of actions, as mentioned previously, may comprise one or both of the second primary list of actions and/or the second secondary list of actions.

A plurality of types of medical visualization devices may be couplable to the monitor device. Each type may be configured for a respective clinical purpose. The types of medical visualization devices may, for example, comprise urology devices, gastro-intestinal devices, pulmonology devices and ENT devices. For example, a first medical visualization device may be a first type configured for a first clinical purpose, a second medical visualization device may be a second device type configured for a second clinical purpose, and/or a third medical visualization device may be a third device type configured for a third clinical purpose. Each of the plurality of types of medical visualization devices may comprise one or more models, which may designate different versions or brands of a particular device or slightly different devices within the same group/type of devices.

The medical visualization device, of which a representation is displayed within the first portion of the graphical user interface, may be a first primary medical visualization device being a first type of a plurality of types of medical visualization devices couplable to the monitor device. The first primary medical visualization device may be of a primary model of a plurality of models of the first type of medical visualization devices.

The monitor device may be actively coupled to the medical visualization device, of which a representation is displayed within the first portion of the graphical user interface. For example, the monitor device may provide the possibility to configure programmable buttons of a device of the same type and model as a medical visualization device being coupled to the monitor device. Alternatively, or additionally, the monitor device may provide the possibility for a user to configure programmable buttons of a device different from the one being coupled to the monitor device.

The monitor device may be adapted to detect one or more initial user inputs. In response to detecting the one or more initial user inputs the monitor device may display the representation of the medical visualization device. The one or more initial user inputs may be detected prior to the monitor device displaying the representation of the medical visualization device. The one or more initial user inputs may include selection of a button configuration indicator (e.g. in a settings menu).

The one or more initial user inputs may be detected with the display, e.g. when the display is a touch sensitive display. Alternatively or additionally, the one or more initial user inputs may be detected with a mouse, a trackpad and/or a keyboard, which may be coupled with the monitor device.

The one or more initial user inputs may comprise a first initial user input. In response to detecting the first initial user input the monitor device may display, within the first portion of the graphical user interface, a plurality of selectable medical visualization devices. The plurality of selectable medical visualization devices may include a first primary selectable medical visualization device corresponding to a first primary medical visualization device and/or a first secondary selectable medical visualization device corresponding to a first secondary medical visualization device.

The first primary medical visualization device may be of a first type of the plurality of types of medical visualization devices couplable to the monitor device. The first primary medical visualization device may be of a primary model of the plurality of models of the first type of medical visualization devices. The first secondary medical visualization device may be of the first type of the plurality of types of medical visualization devices couplable to the monitor device. The first secondary medical visualization device may be of a secondary model of the plurality of models of the first type of medical visualization devices.

The monitor device may, as previously mentioned be actively coupled to a coupled medical visualization device. The monitor device may be adapted to determine model and type of the coupled medical visualization device, e.g. by reading device identifier information from a device identifier of the coupled medical visualization device, as explained previously.

In accordance with the coupled medical visualization device being of the same model and type as the first primary medical visualization device, the monitor device displays the first primary selectable medical visualization device of the plurality of selectable medical visualization devices displayed within the first portion of the graphical user interface in a first mode. The first mode of the first primary selectable medical visualization device indicating that the first primary medical visualization device is currently coupled. Furthermore, the monitor device displays the first secondary selectable medical visualization device in a second mode indicating that the first secondary medical visualization device is currently not coupled.

In accordance with the coupled medical visualization device being of the same model and type as the first secondary medical visualization device, the monitor device displays the first secondary selectable medical visualization device of the plurality of selectable medical visualization devices displayed within the first portion of the graphical user interface in a first mode. The first mode of the first secondary selectable medical visualization device indicating that the first secondary medical visualization device is currently coupled. Furthermore, the monitor device displays the first primary selectable medical visualization device in a second mode indicating that the first primary medical visualization device is currently not coupled.

The one or more initial user inputs may include a second initial user input corresponding to selection of the first primary selectable medical visualization device. In response to detecting the second initial user input the monitor device may display within the first portion of the graphical user interface, the representation of the medical visualization device, wherein the medical visualization device is the first primary medical visualization device. Correspondingly, if the second initial user input had corresponded to selection of the first secondary selectable medical visualization device, the monitor device may, in response to detecting the second initial user input, display within the first portion of the graphical user interface, the representation of the medical visualization device, wherein the medical visualization device is the first secondary medical visualization device.

The monitor device may display a device selection menu within a third portion of the graphical user interface. The device selection menu may be displayed in response to a user input corresponding to selection of a button configuration indicator (e.g. in a settings menu). Alternatively or additionally, the device selection menu may be displayed simultaneously with display of the representation of the medical visualization device within the first portion of the graphical user interface.

The device selection menu may comprise one or more selectable types of medical visualization devices. The one or more selectable types may include a first selectable type corresponding to a first type of the plurality of types of medical visualization devices couplable to the monitor device. The one or more selectable types may include a second selectable type corresponding to a second type of the plurality of types of medical visualization devices couplable to the monitor device. For example, the first selectable type may correspond to gastro-intestinal devices. The second selectable type may correspond to pulmonology devices.

The monitor device may be adapted to detect a primary user input corresponding to selection of the first selectable type of the device selection menu. In response to detecting the primary user input the monitor device may display, within the first portion of the graphical user interface one or more selectable medical visualization devices of the first type. The one or more selectable medical visualization devices may include the first primary selectable medical visualization device corresponding to the first primary medical visualization device, wherein the first primary medical visualization device comprises one or more programmable buttons. The one or more selectable medical visualization devices may include the first secondary selectable medical visualization device corresponding to the first secondary medical visualization device, wherein the first secondary medical visualization device comprises one or more programmable buttons.

As described above with respect to the content displayed in response to the first initial user input. The one or more selectable medical visualization devices displayed in response to detection of the primary user input, may be displayed with indication of a possible coupled device, i.e. in accordance with the coupled medical visualization device being of the same model and type as the first primary medical visualization device, the monitor device may display the first primary selectable medical visualization device of the one or more selectable medical visualization devices displayed within the first portion of the graphical user interface in a first mode. The first mode of the first primary selectable medical visualization device indicating that the first primary medical visualization device is currently coupled. In accordance with the coupled medical visualization device being of the same model and type as the first secondary medical visualization device, the monitor device displays the first secondary selectable medical visualization device of the one or more selectable medical visualization devices displayed within the first portion of the graphical user interface in a first mode. The first mode of the first secondary selectable medical visualization device indicating that the first secondary medical visualization device is currently coupled.

The monitor device may further be adapted to detect a secondary user input (e.g. while displaying the one or more selectable medical visualization devices within the first portion of the graphical user interface) corresponding to selection of the first primary selectable medical visualization device. In response to detecting the secondary user input the monitor device may display within the first portion of the graphical user interface, the representation of the medical visualization device, wherein the medical visualization device is the first primary medical visualization device. Correspondingly, if the secondary user input had corresponded to selection of the first secondary selectable medical visualization device, the monitor device may, in response to detecting the secondary user input, display within the first portion of the graphical user interface, the representation of the medical visualization device, wherein the medical visualization device is the first secondary medical visualization device.

The primary and/or secondary user inputs may be detected before displaying the representation of the medical visualization device within the first portion of the graphical user interface. However, alternatively, primary and/or secondary user inputs may be detected while and/or after displaying the representation of the medical visualization device within the first portion of the graphical user interface, and in response to detecting the secondary user input the monitor device may display within the first portion of the graphical user interface, a representation of another medical visualization device, wherein the another medical visualization device is the first primary medical visualization device or first secondary medical visualization device, in accordance with the selection of the secondary user input.

The primary and/or secondary user inputs may be detected with the display, e.g. when the display is a touch sensitive display. Alternatively or additionally, the primary and/or secondary user inputs may be detected with a mouse, a trackpad and/or a keyboard, which may be coupled with the monitor device.

The monitor device may, e.g. after the user have associated one or more actions with one or more activations of one or more programmable buttons, display the live representation of the image data within the first portion of the graphical user interface. The user may have associated a first action corresponding to capturing a still image with a short press of the first programmable button. The user may have associated a second action corresponding to recording of a video sequence with a long press of the first programmable button or with a short press or long press of the second programmable button.

While displaying the live representation of the image data within the first portion of the graphical user interface, the monitor device detects a first signal corresponding to activation of a programmable button associated with capturing a still image, e.g. a short press of the first programmable button. In response to detection of the first signal, the monitor device may store a first image file corresponding to the image data received when the first signal was detected. Further in response to detection of the first signal, the monitor device may display within the third portion of the graphical user interface a still image representation of a still image corresponding to the stored first image file. The still image representation may comprise an icon indicative of the still image being a still image.

Also while displaying the live representation of the image data within the first portion of the graphical user interface, before or after having detected the first signal, the monitor device detects a second signal corresponding to activation of a programmable button associated with recording of a video sequence, e.g. a long press of the first programmable button, a short press of the second programmable button or a long press of the second programmable button. In response to detection of the second signal the monitor device may store a first video data file corresponding to image data received before the second signal was detected. Further in response to detection of the second signal, the monitor device may display within the third portion of the graphical user interface a video representation corresponding to a frame of the stored first video data file. The video representation may comprise an icon indicative of the stored first video data file being a video recording.

In some examples, an initial detection of the second signal starts the video recording and a subsequent detection of the second signal (i.e. a second activation of the same type of activation of the same programmable button) stops/finishes the video recording. Hence, the storing of the first video data file and display of the video representation may be in response to detection of the subsequent second signal. Also, the first video data file may correspond to image data received between the initial detection of the second signal and the subsequent detection of the second signal. The flexibility in programming buttons provided by the disclosure allows the user to assign any combination of functionalities to different buttons and a plurality of functionalities to one single button. Thus, it is an advantage, as explained above, to indicate to the user which functionalities assigned has been activated. Hence, in accordance with the disclosure, the user can clearly follow what functionality has been activated.

Various exemplary embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

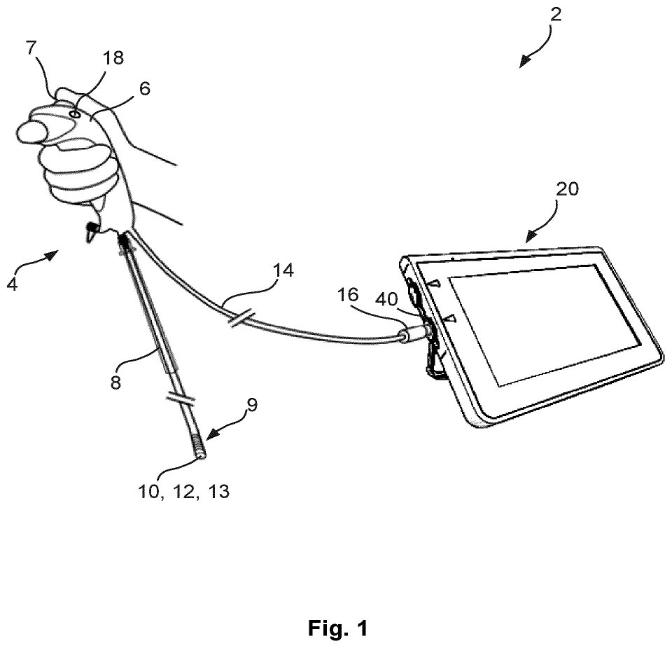

FIG. 1 schematically illustrates an exemplary medical visualization system 2 comprising a medical visualization device 4 and a monitor device 20. The visualization device 4 has an image sensor 12, e.g. a CCD or a CMOS, configured to generate image data indicative of a view from the visualization device 4. In the illustrated example, the visualization device 4 is an endoscope comprising a handle 6 and an insertion cord 8, extending from the handle 6 to a distal cord portion 10. The image sensor 12 may be configured to generate image data indicative of a view from the distal cord portion 10 of the insertion cord 8. The visualization device 4 may further comprise a light emitter 13 adapted to provide illumination of the view. The light emitter 13 may be an LED, an optical fiber connectable to a light source, or similar element known to provide illumination.

The visualization device 4 may be connected to the monitor device 20. In the illustrated example, a device cable 14 extending from the handle 6 terminates in a device connector 16 connected to a connection port 40 of the monitor device 20. The monitor device 20 is operable to receive image data generated by the image sensor 12 of the visualization device 4. For example, the monitor device 20 may receive image data generated by the image sensor 12 via the device cable 14, the connector 16 and connection port 40. Alternatively, the visualization device 4 may be wirelessly connected to the monitor device 20.

The monitor device 20 is configured to display images captured by the medical visualization device 4. The monitor device 20 may comprise a display, such as a touch sensitive display, as illustrated, and/or may be connected to an external display where the images captured by the medical visualization device 4 are displayed.

The handle 6 comprises a control mechanism 7 adapted to receive an input in a first input direction and/or in a second input direction. The touch input in the first input direction on the control mechanism 7 causes a bending section 9 of the insertion cord 8 to bend in a first bending direction, e.g. via wires extending from the handle, through the insertion cord 8 to the bending section 9. The touch input in the second input direction on the control mechanism 7 causes the bending section 9 of the insertion cord 8 to bend in a second bending direction. The first input direction and the second input direction may be opposite. The first bending direction and the second bending direction may be opposite. Bending the bending section 9 of the insertion cord 8 may cause a movement of the distal end 10 and the image sensor 12 in a direction relative to the image sensor 12. Thereby, seeing an image generated by the image sensor 12, a direction, e.g. up or down, in the image may correspond to a respective input on the control mechanism 7.

The handle 6 may have one or more programmable buttons 18. As will be further described below, the programmable button(s) 18 may be configured to execute functions of the monitor device, such as capturing of images, start/stop of video recordings, changing image characteristics, etc. By having one or more programmable buttons on the handle 6, the operator may easily execute desired functionality without having to reach over to tap a button on the monitor device.

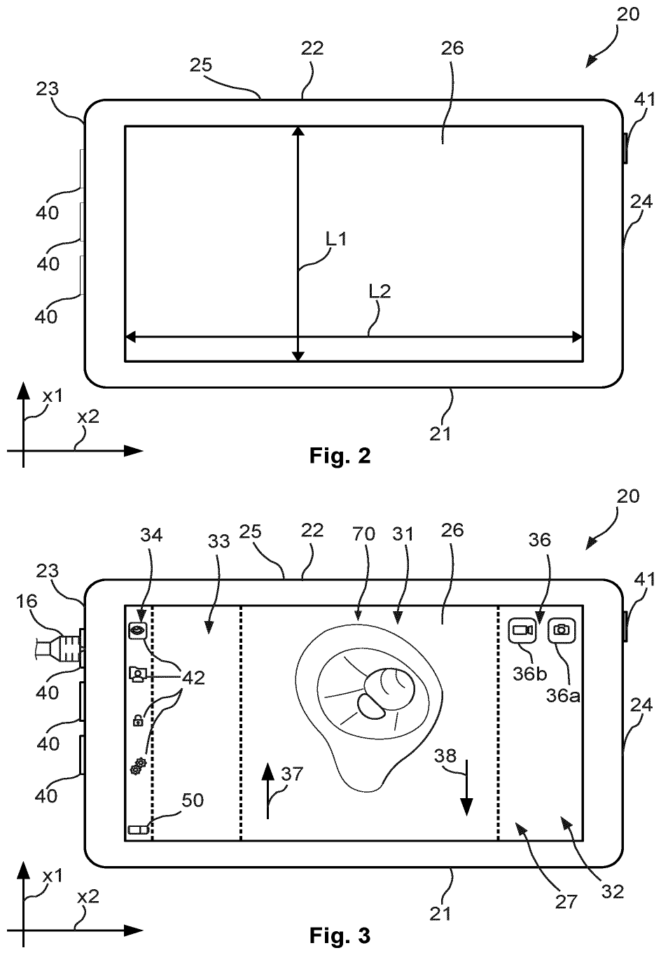

FIG. 2 schematically illustrates an exemplary monitor device 20, such as the monitor device 20 as illustrated in FIG. 1. The monitor device 20 comprises a first housing 25. The first housing 25 extends in a first direction x1 from a first housing side 21 to a second housing side 22 and in a second direction x2 perpendicular to the first direction x1 from a third housing side 23 to a fourth housing side 24. The monitor device comprises a touch sensitive display 26 accommodated in the first housing 25. The touch sensitive display 26 has a first length L1 in the first direction x1 and a second length L2 in the second direction x2. The second length L2 may be longer than the first length L1 as illustrated. In some examples, the monitor device 20 may comprise a second housing (not shown), which does not comprise a display. For example, the second housing may be coupled to the first housing 25, or the second housing may be coupled to an external display.

The monitor device 20 may comprise one or more connection port(s) 40, such as three connection ports 40, as illustrated. The connection ports 40 may allow visualization devices to be connected. The connection port(s) 40 may be arranged at the third housing side 23, as illustrated. Alternatively or additionally, connection port(s) 40 may be arranged at the fourth housing side 24. In some examples, the monitor device 20 may provide for wireless connection with the visualization devices, in which case the connection ports 40 may be omitted. However, in some examples, the monitor device 20 may comprise both connection ports 40 and the ability to wireless connect to visualization devices.

The monitor device may comprise an on/off button 41, which may be provided on the fourth housing side 24, as illustrated.

FIG. 3 schematically illustrates an exemplary monitor device 20, such as the monitor device 20 as illustrated in FIGS. 1-2. As illustrated a device connector 16 may be connected to a connection port 40.

The monitor device 20 may be provided with a graphical user interface 27. The graphical user interface 27 may be displayed with the touch sensitive display 26, and the user may interact with the graphical user interface 27, e.g. by means of providing touch inputs on the touch sensitive display 26. In some examples, the monitor device may be provided without a touch sensitive display, and the user may interact with the graphical user interface by other means, e.g. by a keyboard, a trackpad or a mouse.

The graphical user interface 27 comprises a plurality of non-overlapping portions 31, 32, 33, 34. Each of the portions 31, 32, 33, 34 extends substantially throughout the first length L1 in the first direction x1. The non-overlapping portions includes a first portion 31, a second portion 32, a third portion 33 and a fourth portion 34. The first portion 31 is arranged between the third portion 34 and the second portion 32 along the second direction x2. The third portion 33 is arranged between the fourth portion 34 and the first portion 31 along the second direction x2. The fourth portion 34 is arranged between a side of the first housing, e.g. the third housing side 23, and the third portion 33 along the second direction x2. The second portion 32 is arranged between another side of the first housing 25, e.g. the fourth housing side 24, and the first portion 31 along the second direction x2. The first portion 31 and the third portion 33 are arranged between the second portion 32 and the fourth portion 34 along the second direction x2.

The monitor device 20 displays a live representation 70 of the image data within the first portion 31 of the touch sensitive display 26. The first bending direction and the second bending direction of the bending section 9 of the insertion cord 8, as described with respect to FIG. 1, may corresponds to a first image direction 37 and a second image direction 38 of the live representation 70, respectively. The first image direction 37 and the second image direction 38 may be parallel to the first direction x1, as illustrated. The first image direction 37 and the second image direction 38 may be opposite, as illustrated. Thereby, a user operating the control mechanism 7 of visualization device 4 may cause movement of the bending section 9 of the insertion cord 8 to bend in a direction corresponding to the first image direction 37 or the second image direction 38 of the live representation 70.

The monitor device 20 displays with the touch sensitive display 26 one or more actionable items 36 within the second portion 32 of the graphical user interface 27. The actionable items 36 may comprise an image capture button 36a, e.g. for storing an image data file corresponding to the image data received when the image capture button 36a was activated. Alternatively or additionally, the actionable items 36 may comprise a video capture button 36b, e.g. for storing a video sequence of image data corresponding to the image data received when the video capture button 36b was activated.

The monitor device 20 displays with the touch sensitive display 26 one or more actionable menu items 42 within the fourth portion 34 of the graphical user interface 27. The actionable menu items 42 may, for example, comprise a login menu item for initiating a login procedure, a settings menu item for accessing a settings menu, an archive menu item for browsing an archive, and a default menu item for returning to a default view. Also, a battery indicator 50 is displayed in the fourth portion 34.

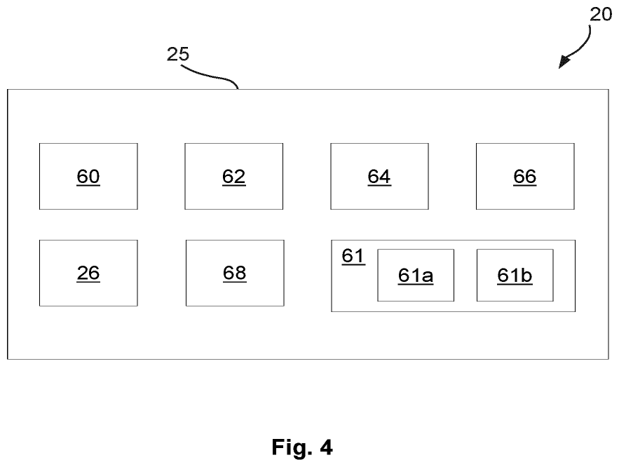

FIG. 4 is a block diagram of an exemplary monitor device 20, such as the monitor device 20 of the previous figures. The monitor device 20 comprises a processing unit 60 and memory 62. The memory 62 may comprise both volatile and non-volatile memory. The monitor device 20 also comprise an orientation sensor 64 for determining the orientation of a housing, such as the first housing 25, relative to gravity. The orientation sensor 64 may comprise one or more accelerometers and/or a gyroscope. The monitor device 20 comprises input/output module 66, such as for receiving image data from the image sensor 12 via connectors of the visualization device 4. The input/output module 66 may also comprise ethernet connector, Wi-Fi transceiver, Bluetooth transceiver, video connectors, USB ports etc., and respective controllers. The monitor device 20 also comprises the touch sensitive display 26 as described earlier. The monitor device 20 may display information, graphical user interface objects, images, buttons etc., with the touch sensitive display 27. The monitor device 20 also comprises a microphone 68. The monitor device 20 comprises a power unit 61 for powering the monitor device 20. The power unit 61 may comprise a rechargeable battery 61a. The power unit 61 may comprise a power connection 61b for connecting the power unit 61 to an external power supply, such as a conventional AC power socket. The components of the monitor device 20 may be interconnected by buses or signal lines. Some or all of the components of the monitor device may be accommodated in the first housing 25 as illustrated. However, alternatively some of the components, e.g. the processing unit 60, the memory 62, input/output module 66 and/or the power unit 61 may be accommodated in a second housing (not shown) of the monitor device 20.

The power unit 61 may comprise components for, e.g. indirectly measuring, capacity of the rechargeable battery 61a. For example, the power unit 61 may comprise a voltage gauge to measure the voltage of the rechargeable battery 61a. Based on the measured voltage, the remaining capacity of the rechargeable battery 61a may be estimated, e.g. by the processing unit 60. The power unit 61 may also comprise components for measuring power consumption of the monitor device 20. For example, the power unit 61 may comprise a power meter to measure the rate at which the monitor device 20 consumes power from the rechargeable battery 61a. The voltage gauge may be a low current consumption integrated circuit or a resistor coupled in parallel with the battery. A current sensor may be provided and the power may be computed as the product of the voltage and current. Additionally, an integrated circuit may be provided that includes a voltage gauge and a current sensor, and which outputs a power value in digital form.

The monitor device 20 may display content with the touch sensitive display 26 and/or with an externally coupled display, which in some examples may be an external touch sensitive display. For example, the monitor device 20 may display content by the processing unit 60 transmitting instructions to the touch sensitive display 26 and/or the externally coupled display indicative of the content to be displayed. The processing unit 60 may be adapted to receive the image data from the visualization device. The processing unit 60 may be adapted to cause a display, such as the touch sensitive display 26 and/or the externally coupled display, to display a live representation of the image data.

The monitor device 20 may receive user input with the touch sensitive display 26. Particularly, the monitor device 20 may detect user inputs with the touch sensitive display 26. For example, a user providing a touch input on the touch sensitive display 26 causes a change in one or more electrical parameters of the touch sensitive display 26 indicative of at least the location of the touch input. Information of the touch input is transmitted from the touch sensitive display 26 to the processing unit 60, and the processing unit 60 may determine whether the touch input corresponds to an action to perform, e.g. whether the location of the touch input corresponds to the location of a soft-button displayed at the touch sensitive display. Alternatively or additionally, the monitor device 20 may be receive user input by other means, e.g. by a keyboard, a trackpad or a mouse, which may be coupled to the monitor device 20. As will be described later, the monitor device 20 may also or alternatively receive user input by means of buttons on a coupled visualization device.

The user may interact with the monitor device 20 via the graphical user interface 27 by providing user inputs, e.g. by means of providing touch inputs on the touch sensitive display 26, and the monitor device 20 may detect such user inputs with the touch sensitive display 26. A touch input, e.g. a single tap, long press, double tap, swipe or similar, and the location of the touch input on the touch sensitive display 26 is registered by the touch sensitive display 26, which transmits information of the touch input (e.g. including type of touch (double tap, long press, single tap, swipe, etc.) and/or location of the touch) to the processing unit 60 of the monitor device 20. The processing unit 60 interprets the information received and determines whether the touch input corresponds to activation of an action, e.g. whether the touch input correspond to activation of a button displayed with the touch sensitive display 27 at the location of the touch input. In response to a determination that the touch input corresponds to activation of an action, the processing unit 60 performs the respective action.

For example, with reference to FIGS. 3 and 4, to capture an image corresponding to the presently shown live representation 70, e.g. corresponding to the image data received from the image sensor, the user may tap the image capture button 36a. The tap and the location of the tap is registered by the touch sensitive display 26, which transmits the information of the tap to the processing unit 60 of the monitor device 20. The processing unit 60 interprets the information received and determines that the user tapped the location corresponding to the image capture button 36a. In response thereto, the processing unit 60 stores, in memory 62 an image data file corresponding to the image data received.

In further reference to FIGS. 3 and 4, to capture a video sequence corresponding to the shown live representation 70 over a period of time, e.g. corresponding to the image data received from the image sensor over a period of time, the user may tap the video capture button 36b. The tap and the location of the tap is registered by the touch sensitive display 26, which transmits the information of the tap to a processing unit 60 (see FIG. 4) of the monitor device 20. The processing unit 60 interprets the information received and determines that the user tapped the location corresponding to the video capture button 36b. In response thereto, the processing unit 60 starts collection of image data received from the image sensor 12 and temporarily stores the data in memory 62. To stop the recording, the user may tap the video capture button 36b again. The processing unit 60 determines, based on the signal received from the touch sensitive display 26, that that the user tapped the video capture button 36b and stops collecting image data received from the image sensor 12. The processing unit 60 read the temporarily stored data from the memory 62 and create a complete video sequence based thereon, which the processing unit 60 stores in the memory 62.

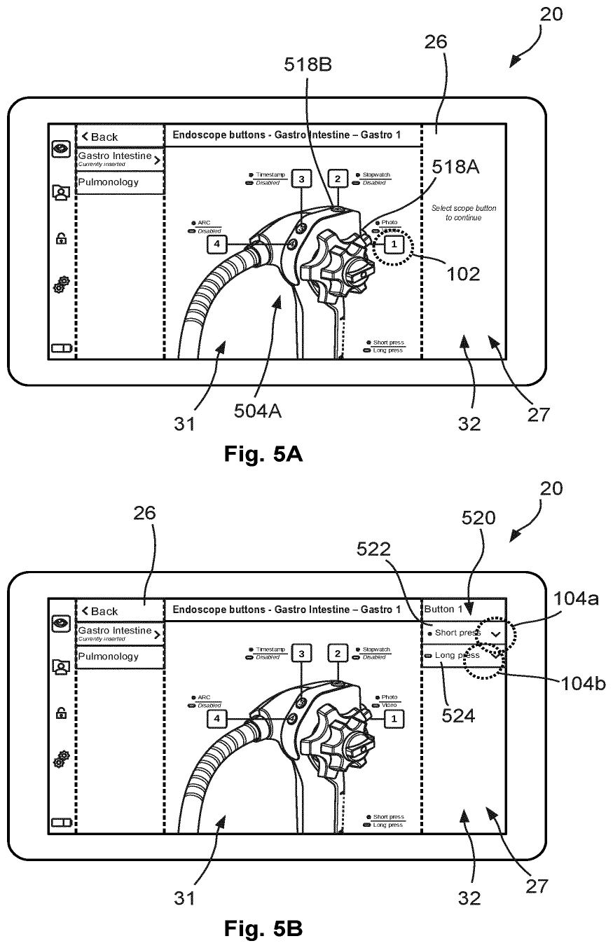

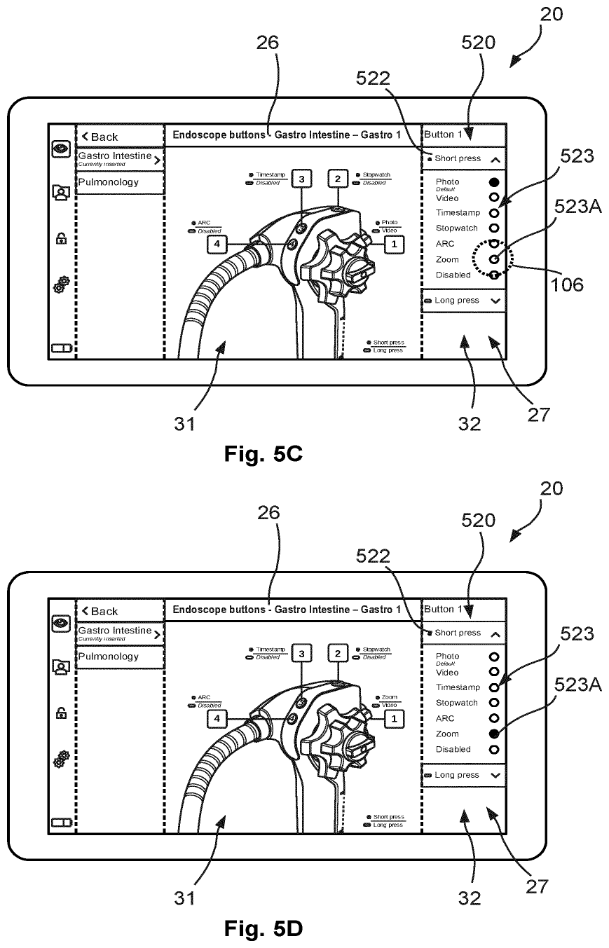

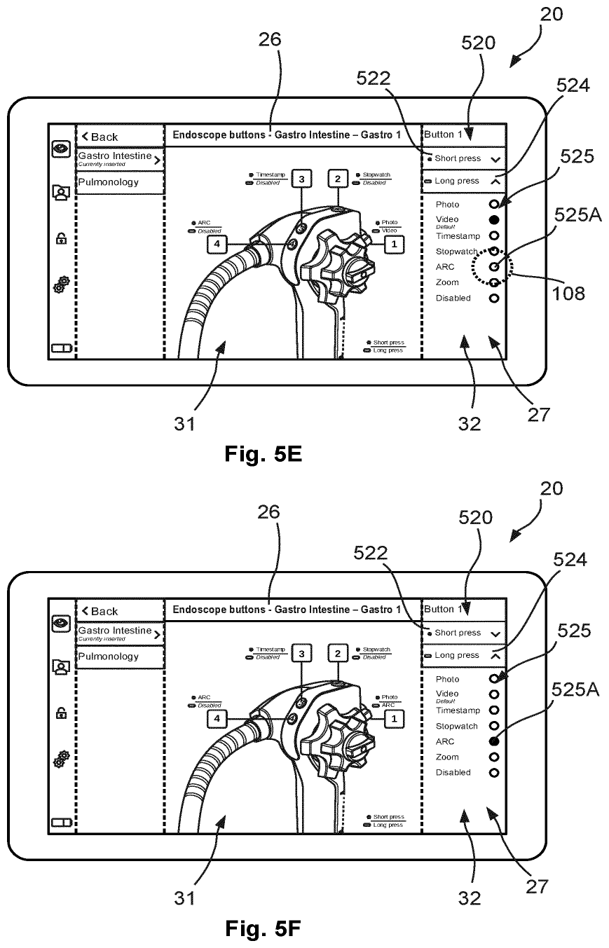

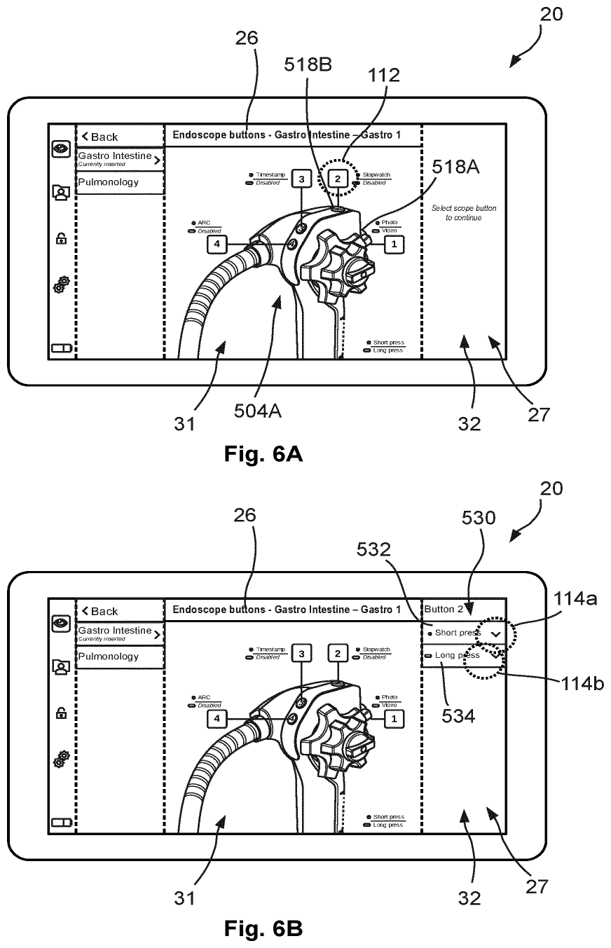

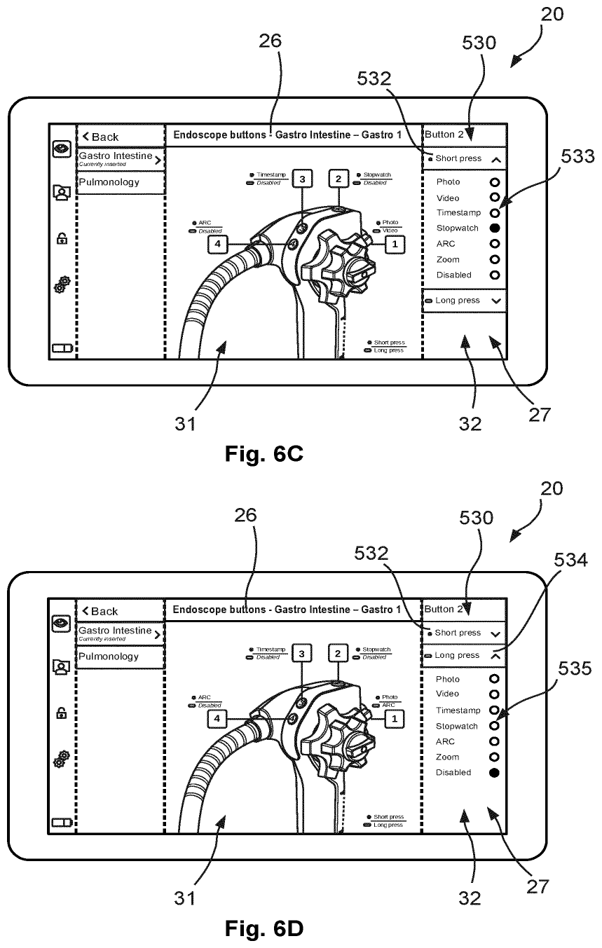

FIGS. 5A-5F and 6A-6D schematically illustrates exemplary user interactions with an exemplary graphical user interface 27 of a monitor device 20, such as the monitor device 20 and the graphical user interface 27, as described previously. Particularly, FIGS. 5A-5F and 6A-6D schematically illustrates an exemplary part of the graphical user interface 27 for setting up programmable buttons of a medical visualization device, such as the visualization device 4, as described in relation to FIG. 1.

As illustrated in FIG. 5A, a representation of a medical visualization device 504A is displayed within the first portion 31 of the graphical user interface 27. The medical visualization device 4 comprises a plurality of programmable buttons including a first programmable button 518A and a second programmable button 518B.

The monitor device 20 is adapted to detect a first user input 102 corresponding to selection of the first programmable button 518A. In response to detecting the first user input 102, as illustrated in FIG. 5B the monitor device 20 displays, within the second portion 32 of the graphical user interface 27, a first button setup menu 520. The first button setup menu 520 is for selecting one or more actions to be associated with one or more activations of the first programmable button 518A. The heading of the first button setup menu 520 illustrates that the first button setup menu 520 is for selecting actions to be associated with the first programmable button 518A.

A button may be assigned different actions depending on the type of activation. For example, monitor device 20 and/or the medical visualization device 504A may be able to distinguish between a primary activation, e.g. a short press, and a secondary activation, e.g. a long press. Hence, each button may potentially be assigned two actions. A short press of a programmable button, such as the first programmable button 518A and/or the second programmable button 518B may correspond to an activation of the respective button for a duration being less than a threshold duration (e.g. 1 second), and the long press may correspond to an activation of the respective button for a duration being longer than the threshold duration.

Accordingly, in the illustrated example, the first button setup menu 520 comprises a first short press menu option 522 and a first long press menu option 524. The first short press menu option 522 is for selecting a first primary action to be associated with the short press of the first programmable button 518A. The first long press menu option 524 is for selecting a first secondary action to be associated with the long press of the first programmable button 518A. The user may choose the same action to be associated both with the long press and the short press, and/or the user may choose to disable actions for the long press or the short press of a button.

In the illustrated example, an overview visually illustrating the actions assigned to the different activations (long press and short press) of each of the programmable buttons is also displayed within the first portion 31 of the graphical user interface 27 in connection with the representation of the medical visualization device 504A. For example, as illustrated, the first programmable button 518A has its short press assigned to capturing a still image and having its long press assigned to starting/stopping a video recording. Short press functions of the programmable buttons may be indicated by a short, e.g. circular, indicator, as illustrated. This indicator for the short press functions of the programmable buttons may be the same for each button shown in the first portion 31 of the graphical user interface 27, and for the short press menu option 522 displayed in the second portion 32 of the graphical user interface 27. Long press functions of the programmable buttons may be indicated by an elongated indicator, as illustrated. This indicator for the long press functions of the programmable buttons may be the same for each button shown in the first portion 31 of the graphical user interface 27, and for the long press menu option 524 displayed in the second portion 32 of the graphical user interface 27.

In FIG. 5B, the first short press menu option 522 and the first long press menu option 524 of the first button setup menu 520 are collapsed. The first short press menu option 522 may be expanded by user input 104a. The first long press menu option 524 may be expanded by user input 104b. FIG. 5C illustrates the expanded first short press menu option 522, e.g. caused by user input 104a as illustrated in FIG. 5B. The first short press menu option 522 comprises a first primary list of actions 523. In the illustrated example, the short press of the first programmable button 518A is presently assigned to the action “photo”, which causes capturing of a still image. The presently assigned action is indicated by a filled circle, while other actions, which the user may assign is indicated by an open circle.

The user may select a first primary action 523A (e.g. “zoom” in the illustrated example) to be associated with the short press of the first programmable button 518A by providing a user input 106, such as a tap on the touch sensitive display 26, corresponding to the first primary action 523A. By selecting the first primary action 523A by the user input 106, the first primary action is associated with the short press of the first programmable button 518A, as illustrated in FIG. 5D. After selecting the first primary action 523A, this action is indicated by a filled circle, and the previously associated action is indicated with an open circle, like the other actions of the first primary list of actions 523. The overview within the first portion 31 of the graphical user interface 27 in connection with the representation of the medical visualization device 504A is also updated to reflect the newly assigned action of the first programmable button 518A.

FIG. 5E illustrates the expanded first long press menu option 524, e.g. caused by user input 104b as illustrated in FIG. 5B. The first long press menu option 524 comprises a first secondary list of actions 525. The first primary list of actions 523, as illustrated in FIG. 5C, and the first secondary list of actions 525, as illustrated in FIG. 5E, may comprise the same actions. Hence, the user may be able to select between the same actions for assigning to either of the activations (e.g. short press or long press) of the first programmable button.

In the illustrated example, the long press of the first programmable button 518A is presently assigned to the action “video”, which causes start or stop of a video recording. The presently assigned action is indicated by a filled circle, while other actions, which the user may assign is indicated by an open circle.

The user may select a first secondary action 525A (e.g. “ARC” in the illustrated example) to be associated with the long press of the first programmable button 518A by providing a user input 108, such as a tap on the touch sensitive display 26, corresponding to the first secondary action 525A. By selecting the first secondary action 525A by the user input 108, the first secondary action 525A is associated with the long press of the first programmable button 518A, as illustrated in FIG. 5F. After selecting the first secondary action 525A, this action is indicated by a filled circle, and the previously associated action is indicated with an open circle, like the other actions of the first secondary list of actions 525. The overview within the first portion 31 of the graphical user interface 27 in connection with the representation of the medical visualization device 504A is also updated to reflect the newly assigned action of the first programmable button 518A.

FIG. 6A illustrates the same as FIG. 5A. However, in FIG. 6A the monitor device detects a second user input 112 corresponding to selection of the second programmable button 518B. In response to detecting the second user input 112 the monitor device 20 displays, within the second portion 32 of the graphical user interface 27, a second button setup menu 530. The second button setup menu 530 is for selecting one or more actions to be associated with one or more activations of the second programmable button 518B. The heading of the second button setup menu 530 illustrates that the second button setup menu 530 is for selecting actions to be associated with the second programmable button 518B.

In the illustrated example, the second button setup menu 530 comprises a second short press menu option 532 and a second long press menu option 534. The second short press menu option 532 is for selecting a second primary action to be associated with the short press of the second programmable button 518B. The second long press menu option 534 is for selecting a second secondary action to be associated with the long press of the second programmable button 518B.

In the illustrated example, the second short press menu option 532 and the second long press menu option 534 of the second button setup menu 530 are collapsed. The second short press menu option 532 may be expanded by user input 114a. The second long press menu option 534 may be expanded by user input 114b.

FIG. 6C illustrates the expanded second short press menu option 532, e.g. caused by user input 114a as illustrated in FIG. 6B. The second short press menu option 532 comprises a second primary list of actions 533. The second primary list of actions 533, as illustrated in FIG. 6C and the first primary list of actions 523, as illustrated in FIG. 5C, may comprise the same actions. Hence, the user may be able to select between the same actions for assigning to either of the buttons and may potentially assign several buttons to the same action.

FIG. 6D illustrates the expanded second long press menu option 534, e.g. caused by user input 114b as illustrated in FIG. 6B. The second long press menu option 534 comprises a second secondary list of actions 535. The second secondary list of actions 535, as illustrated in FIG. 6D, may comprise the same actions as the second primary list of actions 533, as illustrated in FIG. 6C.

The actions associated to the activations of the second programmable button 518B may be changed similarly as described in relation to the first programmable button 518A.

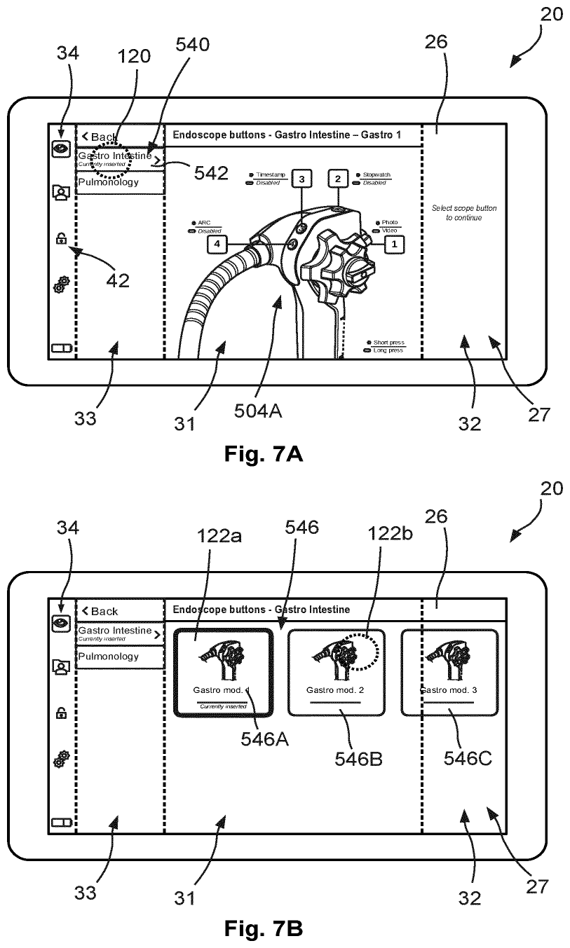

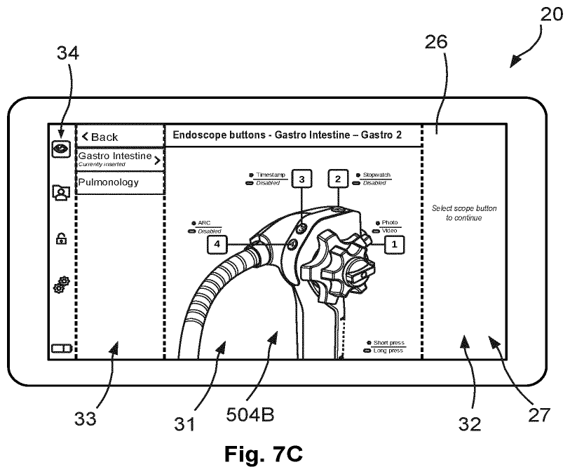

FIGS. 7A-7C schematically illustrates exemplary user interactions with an exemplary graphical user interface 27 of a monitor device 20, such as the monitor device 20 and the graphical user interface 27, as described previously. Particularly, FIGS. 7A-7C schematically illustrates an exemplary part of the graphical user interface 27 for setting up programmable buttons of a medical visualization device, such as the visualization device 4, as described in relation to FIG. 1, and more specifically for changing the type of medical visualization device for configuring the programmable buttons.

Different medical visualization devices may be couplable to the monitor device 20. Thus, it may be advantageous to be able to change to setup of a different visualization device, which may comprise a different number or layout of programmable buttons.

A plurality of types of medical visualization devices may be couplable to the monitor device 20. The types of medical visualization devices may, for example, comprise urology devices, gastro-intestinal devices, pulmonology devices and ENT devices. Each of the plurality of types of medical visualization devices may comprise one or more models, which may designate different versions or brands of a particular device or slightly different devices within the same group/type of devices.

The button setup, as described with respect to FIGS. 5A-6D, may as default, be provided for a type and model similar to a medical visualization device actively coupled to the monitor device 20.

The monitor device 20 may be adapted to detect one or more initial user inputs 120, 122a, 122b (as illustrated in FIGS. 7A-7B), and in response to detecting the one or more initial user inputs 120, 122, the monitor device 20 displays the button setup as illustrated and described in relation to FIGS. 5A-6D corresponding to the medical visualization device selected by the initial user inputs 120, 122.

As illustrated in FIG. 7A, a device selection menu 540 may be displayed within the third portion 33 of the graphical user interface 27. The device selection menu 540 comprises selectable types of medical visualization devices including a first selectable type 542 corresponding to a first type of the plurality of types of medical visualization devices couplable to the monitor device. And as illustrated in FIG. 7A, the one or more initial user inputs may comprise a first initial user input 120 (alternatively denoted “primary user input”) corresponding to selection of the first selectable type 542 (“Gastro Intestine” in the illustrated example). The selectable types may correspond to respective types configured for respective clinical purpose. For example, the first selectable type 542 may be a first type configured for a first clinical purpose, such as gastroenterology. A second selectable type of the selectable types of the device selection menu 540 may be a second type configured for a second clinical purpose, such as pulmonology.

The monitor device 20 may be actively coupled to a coupled medical visualization device and may be adapted to determine model and type of the coupled medical visualization device. For example, the coupled medical visualization device may comprise an electronically readable memory (e.g. an EPROM), which may be read by the processing unit of the monitor device 20. Based on the information read from the memory of the coupled medical visualization device, the monitor device 20 may be able to determine model and type of the coupled medical visualization device.

The coupled medical visualization device may be of the first type as indicated by the first selectable type 542. Accordingly, the monitor device 20 may display the first selectable type 542 in the device selection menu 540 with an indication, e.g. a label stating: “Currently inserted”, that a device of the first selectable type 542 is currently coupled.

Hence the user can easily setup one or more medical visualization devices if they are coupled to the monitor device since the clinical purpose may be selected in device selection menu 540. Furthermore, because of the labeling of the type of the currently coupled medical visualization device, the user is guided towards the setup of the relevant medical visualization device, even if two different medical visualization devices for different clinical purposes have to be configured.

As illustrated, the device selection menu 540 may be displayed together with the button setup procedure of a particular medical visualization device, such as the first primary medical visualization device 504A, as illustrated. However, the device selection menu 540 may also be accessible while displaying other content within the first portion 31 and/or the second portion 32 of the graphical user interface 27. For example, the device selection menu 540 may be accessible via a settings menu item among the actionable menu items 42 within the fourth portion 34 of the graphical user interface 27.

In response to detecting the first initial user input 120 the monitor device 20 displays, as illustrated in FIG. 7B, a plurality of selectable medical visualization devices 546, within the first portion 31 of the graphical user interface 27. The plurality of selectable medical visualization devices 546 may extend into the second portion 32 of the graphical user interface 27. The plurality of selectable medical visualization devices 546 includes a first primary selectable medical visualization device 546A corresponding to a first primary medical visualization device. The plurality of selectable medical visualization devices 546 includes a first secondary selectable medical visualization device 546B corresponding to a first secondary medical visualization device. In the illustrated example, the plurality of selectable medical visualization devices 546 also includes a first tertiary selectable medical visualization device 546C.

The monitor device 20 may be actively coupled to a coupled medical visualization device, and may be adapted to determine model and type of the coupled medical visualization device. For example, the coupled medical visualization device may comprise an electronically readable memory (e.g. an EPROM), which may be read by the processing unit of the monitor device 20. Based on the information read from the memory of the coupled medical visualization device, the monitor device 20 may be able to determine model and type of the coupled medical visualization device.

The coupled medical visualization device may be of the same model and type as the first primary medical visualization device as indicated by the first primary selectable medical visualization device 546A as illustrated in FIG. 7B. Accordingly, the monitor device 20 displays the first primary selectable medical visualization device 546A in a first mode indicating that the first primary medical visualization device is currently coupled. For example, as illustrated the first primary selectable medical visualization device 546A is shown with a bold border and with a label stating: “Currently inserted”.

The monitor device 20 displays the first secondary selectable medical visualization device, e.g. since a medical visualization device of that type and model is not currently coupled to the monitor device 20, in a second mode indicating that the first secondary medical visualization device is currently not coupled. For example, as illustrated, the first secondary selectable medical visualization device 546B is illustrated with a thin border, and without the label stating: “Currently inserted”.

It will be understood that in the situation where the coupled medical visualization device was of the same model and type as the first secondary medical visualization device, the monitor device 20 would display the first secondary selectable medical visualization device 546B in the first mode indicating that the first secondary medical visualization device was currently coupled. The monitor device would further display the first primary selectable medical visualization device in the second mode indicating that the first primary medical visualization device was currently not coupled.

The monitor device 20 might in some examples be simultaneously coupled to a plurality of medical visualization devices, in which case more than one of the selectable medical visualization devices 546 may be displayed in the first mode.

A second initial user input 122a, 122b (alternatively denoted “secondary user input”) corresponding to selection of a selectable medical visualization device may be detected, and in response thereto the monitor device displays within the first portion 31 of the graphical user interface 27, a representation of the corresponding medical visualization device. For example, in response to the second initial user input 122a corresponding to selection of the first primary selectable medical visualization device, the monitor device 20 displays the graphical user interface as exemplified in FIG. 5A, i.e. displaying the representation of the first primary medical visualization device 504A within the first portion 37. In response to the second initial user input 122b corresponding to selection of the first secondary selectable medical visualization device (i.e. a different type and model of medical visualization device), the monitor device 20 displays a representation of the first secondary medical visualization device 504B within the first portion 37, as illustrated in FIG. 7C.

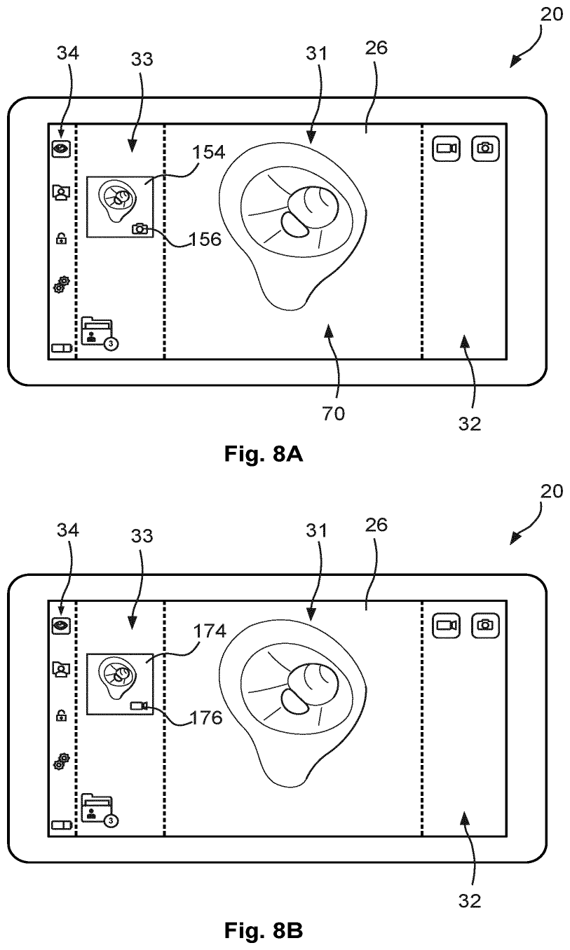

FIGS. 8A and 8B schematically illustrates exemplary part of a graphical user interface 27, such as the graphical user interface 27 as also described in relation to FIGS. 5A-7C. Particularly, FIGS. 8A and 8B shows examples wherein a user has activated an action, e.g. by activation of one of the programmable buttons of the medical visualization device, as explained above, which corresponds to storing of an image file (FIG. 8A) or storing of a video sequence (FIG. 8B) corresponding to the image data received when the activation was detected, e.g. corresponding to the live representation 70 being displayed in the first portion 31.

For example, a short press of the first programmable button may have been assigned to the action corresponding to capturing of a still image. A long press of the first programmable button may have been assigned to the action corresponding to recording of a video sequence. In some examples, a short press and long press of a single programmable button may be configured to execute interrelated actions of a single function. For example, the short press and long press of the single programmable button may be configured to respectively start and stop a function, such as a video recording.

Accordingly, in response to detection of a short press of the first programmable button, the monitor device stores an image file corresponding to the image data received (and displayed as the live representation 70) when the short press of the first programmable button was detected. Additionally, as illustrated in FIG. 8A, the monitor device displays within the third portion 33 of the graphical user interface 27, an image representation 154 of a still image corresponding to the stored image file. To indicate that a still image was captured (and not a video recording) the image representation 154 comprises an image icon 156, indicative of a still image.

Similarly, in response to detection of a long press of the first programmable button, the monitor device stores a video file corresponding to a sequence of image data received (and displayed as the live representation 70) before or after the long press of the first programmable button was detected. It is noted that the long press causing storing of the video file may be a second long press, as a first long press may start the video recording and the second long press may stop and save the video recording. Additionally, as illustrated in FIG. 8B, the monitor device displays within the third portion 33 of the graphical user interface 27, a representation 174 of a frame of the stored video file. To indicate that a video recording was captured (and not a still image) the representation 174 comprises a video icon 156, indicative of a video recording.

By providing icons 156, 176 on the thumbnails, the user may more easily recognize what action he/she has activated, which may be particularly advantageous when buttons may be programmed as per each user's preference and when each button may activate different (although in some examples closely related) functionality depending on the type of activation, e.g. long press or short press.

The disclosure has been described with reference to a preferred embodiment. However, the scope of the invention is not limited to the illustrated embodiment, and alterations and modifications can be carried out without deviating from the scope of the invention.

Throughout the description, the use of the terms “first”, “second”, “third”, “fourth”, “primary”, “secondary”, “tertiary” etc. does not imply any particular order or importance but are included to identify individual elements. Furthermore, the labelling of a first element does not imply the presence of a second element and vice versa.

### LIST OF REFERENCES

2 medical visualization system
4 visualization device
6 handle
7 control mechanism
8 insertion cord
9 bending section
10 distal cord portion
12 image sensor
13 light emitter
14 device cable
16 device connector
18 programmable button
20 monitor device
21 first housing side
22 second housing side
23 third housing side
24 fourth housing side
25 first housing
26 touch sensitive display
27 graphical user interface
31 first portion
32 second portion
33 third portion
34 fourth portion
36 actionable item(s)
37 first image direction
38 second image direction
40 connection port(s)
42 actionable menu item(s)
50 battery indicator
60 processing unit
61 power supply
61a battery
61b power connection
62 memory
64 orientation sensor
66 input/output
68 microphone
70 live representation of image data
x1 first direction
x2 second direction
L1 first length
L2 second length
102, 104, 106, 108, 112, 114a, 114b, 116, 120, 122a, 122b user input
504A, 504B medical visualization device
518A, 518B programmable button
520 first button setup menu
522 first short press menu option
523 first primary list of actions
523A first primary action
524 first long press menu option
525 first secondary list of actions
525A first secondary action
530 second button setup menu
532 second short press menu option
533 second primary list of actions
533A second primary action
534 second long press menu option
535 second secondary list of actions
535A second secondary action
540 device selection menu
542 first selectable type
546 selectable medical visualization devices
546A, 546B, 546C selectable medical visualization device

1. A monitor device operable to receive image data from one or more medical visualization devices, the monitor device comprising:
a housing, and
a processing unit adapted to receive the image data from the medical visualization device and cause a display to display a live representation of the image data,
wherein the monitor device displays with the display a graphical user interface and displays, within a first portion of the graphical user interface, a representation of a medical visualization device comprising one or more programmable buttons including a first programmable button, and
wherein the monitor device is adapted to detect a first user input corresponding to selection of the first programmable button, and in response to detecting the first user input the monitor device displays, within a second portion of the graphical user interface, a first button setup menu for selecting one or more actions to be associated with one or more activations of the first programmable button.

2. The monitor device of claim 1, wherein the one or more activations of the first programmable button includes a short press of the first programmable button and a long press of the first programmable button, and wherein the first button setup menu comprises a first short press menu option for selecting a first primary action to be associated with the short press of the first programmable button and a long press menu option for selecting a first secondary action to be associated with the long press of the first programmable button.

3. The monitor device of claim 2, wherein the short press of the first programmable button corresponds to an activation of the first programmable button for a duration being less than a threshold duration, and wherein the long press of the first programmable button corresponds to an activation of the first programmable button for a duration longer than the threshold duration.

4. The monitor device of claim 2, wherein the first short press menu option comprises a first primary list of actions, and wherein selecting the first primary action to be associated with the short press of the first programmable button includes selecting the first primary action from the first primary list of actions.

5. The monitor device of claim 4, wherein the first long press menu option comprises a first secondary list of actions, and wherein selecting the first secondary action to be associated with the long press of the first programmable button includes selecting the first secondary action from the first secondary list of actions.

6. The monitor device of claim 5, wherein the first primary list of actions and the first secondary list of actions comprise the same actions.

7. The monitor device of claim 1, wherein the one or more programmable buttons of the medical visualization device includes a second programmable button, and wherein the monitor device is adapted to detect a second user input corresponding to selection of the second programmable button, and in response to detecting the second user input the monitor device displays, within the second portion of the graphical user interface, a second button setup menu for selecting one or more actions to be associated with one or more activations of the second programmable button.

8. The monitor device of claim 7, wherein the first button setup menu comprises a first list of actions and the second button setup menu comprises a second list of actions, and wherein the first list of actions and the second list of actions comprises the same actions.

9. The monitor device of claim 1, wherein the medical visualization device is a first primary medical visualization device being a first type of a plurality of types of medical visualization devices couplable to the monitor device, and the first primary medical visualization device being of a primary model of a plurality of models of the first type of medical visualization devices.

10. The monitor device of claim 1, wherein the monitor device is actively coupled to the medical visualization device.

11. The monitor device of 1, wherein the monitor device is adapted to detect one or more initial user inputs, and in response to detecting the one or more initial user inputs the monitor device displays the representation of the medical visualization device.

12. The monitor device of claim 11, wherein the one or more initial user inputs comprises a first initial user input and in response to detecting the first initial user input the monitor device displays, within the first portion of the graphical user interface, a plurality of selectable medical visualization devices including a first primary selectable medical visualization device corresponding to a first primary medical visualization device and a first secondary selectable medical visualization device corresponding to a first secondary medical visualization device.

13. The monitor device of claim 12, wherein the monitor device is actively coupled to a coupled medical visualization device, and adapted to determine model and type of the coupled medical visualization device, and wherein:
in accordance with the coupled medical visualization device being of the same model and type as the first primary medical visualization device, the monitor device displays the first primary selectable medical visualization device in a first mode indicating that the first primary medical visualization device is currently coupled, and the monitor device displays the first secondary selectable medical visualization device in a second mode indicating that the first secondary medical visualization device is currently not coupled, and
in accordance with the coupled medical visualization device being of the same model and type as the first secondary medical visualization device, the monitor device displays the first secondary selectable medical visualization device in a first mode indicating that the first secondary medical visualization device is currently coupled, and the monitor device displays the first primary selectable medical visualization device in a second mode indicating that the first primary medical visualization device is currently not coupled.

14. The monitor device of claim 13, wherein the one or more initial user input includes a second initial user input corresponding to selection of the first primary selectable medical visualization device, and in response to detecting the second initial user input the monitor device displays within the first portion of the graphical user interface, the representation of the medical visualization device, wherein the medical visualization device is the first primary medical visualization device.

15. The monitor device of claim 1, wherein the monitor device;
displays a device selection menu within a third portion of the graphical user interface, the device selection menu comprises one or more selectable types of medical visualization devices including a first selectable type corresponding to a first type of a plurality of types of medical visualization devices couplable to the monitor device,
is adapted to detect a primary user input corresponding to selection of the first selectable type of the device selection menu, and in response to detecting the primary user input the monitor device displays, within the first portion of the graphical user interface, one or more selectable medical visualization devices of the first type including a first primary selectable medical visualization device corresponding to a first primary medical visualization device, wherein the first primary medical visualization device comprises one or more programmable buttons, and
is adapted to detect a secondary user input corresponding to selection of the first primary selectable medical visualization device, and in response to detecting the secondary user input the monitor device displays within the first portion of the graphical user interface, the representation of the medical visualization device, wherein the medical visualization device is the first primary medical visualization device.

16. A medical visualization system comprising the monitor device of claim 1 and a medical visualization device, the medical visualization device comprising:
an image sensor adapted to generate image data indicative of a view from the medical visualization device, and
a handle having one or more programmable buttons.

17. The medical visualization system of claim 16, wherein the medical visualization device is a laryngoscope.

18. The medical visualization system of claim 16, wherein the medical visualization device comprises an insertion cord extending from the handle to a distal cord portion, and wherein the view from the visualization device is a view from the distal cord portion of the insertion cord.

19. The medical visualization system of claim 18, wherein the handle comprises a control mechanism adapted to receive an input in a first input direction, and wherein the input in the first input direction causes a bendable section of the insertion cord to bend in a first bending direction.

20. The medical visualization system of claim 16, wherein the medical visualization device is a first primary medical visualization device being a first type of a plurality of types of medical visualization devices couplable to the monitor device, and the first primary medical visualization device being of a primary model of a plurality of models of the first type of medical visualization devices.
